(19) United States
(12) Patent Application Publication
(10) Pub. No.: US 2025/0169891 A1
(43) Pub. Date: May 29, 2025

(54) TRACKING OF OFFSET REAMER HANDLE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Samuel C. Dumpe, Sewickley, PA (US); Branislav Jaramaz, Pittsburgh, PA (US); Eric Ashuckian, Pittsburgh, PA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/955,383

(22) Filed: Nov. 21, 2024

Related U.S. Application Data
(60) Provisional application No. 63/603,790, filed on Nov. 29, 2023.

(51) Int. Cl. A61B 34/20; A61B 17/00; A61B 17/16

(52) U.S. Cl. CPC A61B 34/20; A61B 17/164; A61B 2017/00738; A61B 2034/2051; A61B 2034/2055; A61B 2034/2072

(57) ABSTRACT

An offset reamer coupled with a tracking array having one or more targets for a tracking system. In some examples, the tracking array may be moved to multiple different mounting positions about the axis of reaming at the reamer element or reamer dome. In some examples, the tracking array may be moved between multiple different mounting positions on the offset reamer wherein the current mounting position is known and the translation between the current mounting position and the reamer element is known to the computer system for the tracking system.

### CROSS-REFERENCE TO RELATED APPLICATIONS

This application claims priority under 35 U.S.C. § 119 to a provisional patent application, U.S. Provisional Patent Application Ser. No. 63/603,790 filed on Nov. 29, 2023. The contents of the aforementioned provisional patent application is incorporated herein by reference in its entirety.

### FIELD OF THE DISCLOSURE

The present disclosure relates generally to orthopedic devices and methods and more particularly to an instrumentation set including an offset reamer with an optical or electromagnetic tracking array.

### BACKGROUND OF THE DISCLOSURE

Orthopedic fixation devices (implants) may be used, for example, to stabilize an injury, to support a bone fracture, to fuse a joint, and/or to correct a deformity. Orthopedic fixation devices may be attached permanently or temporarily, and may be attached to the bone at various locations, including implanted within a canal or other cavity of the bone. Some orthopedic fixation devices allow the position and/or orientation of two or more bone pieces, or two or more bones, to be adjusted relative to one another. Orthopedic fixation devices are generally machined or molded from isotropic materials, such as metals including, for example, titanium, titanium alloys, stainless steel, cobalt-chromium alloys, and tantalum.

An intramedullary (“IM”) nail is one type of orthopedic fixation device. The primary function of the IM nail is to stabilize the fracture fragments, and thereby enable load transfer across the fracture site while maintaining anatomical alignment of the bone. Currently, there are a large number of different commercially available IM nails in the marketplace.

In use, an IM nail is arranged and configured to be inserted into the intramedullary canal of a patient's bone, such as, for example, a patient's femur. In preparing a patient's femur to receive an IM nail, the patient may be positioned in the supine or lateral position. Next, an incision may be made and an optimal entry point may be identified. Instrumentation including, for example, an outer tube or sleeve, a handle, and a reamer may be used to ream the patient's intramedullary canal to receive the IM nail.

However, in use, utilization of reamers when inserting an IM nail via a direct anterior approach has been avoided. Current techniques utilizing a direct anterior approach typically rely on broaches to prepare the intramedullary canal of the patient's femur. This is, in part, due to, for example, obstruction caused by the patient's soft tissue. As a result, surgical procedures for preparing and inserting IM nails using a reaming approach are often performed using a posterior-lateral surgical approach.

In addition, during a revision procedure, reaming the intramedullary canal of the patient's femur for insertion of a longer revision IM nail while the patient is in a supine position involves hyperextending and externally rotating the patient's hip in order to gain sufficient access to the patient's proximal femur. This technique can also involve extending the incision and performing an extended trochanteric osteotomy depending on the condition of the primary femoral component. After gaining sufficient exposure, reaming instruments may be used as in a standard surgical workflow with a higher degree of difficulty especially in larger patients when inserting longer femoral reamers and drivers.

An offset reamer handle may enable surgeons to curve or offset the reamer and driver construct around the proximal femoral anatomy to enable surgeons to utilize a direct anterior approach when inserting an IM nail into the patient's femur via reaming (i.e., allow implantation of an IM nail into a patient's femur via a direct anterior approach where the patient's femur is prepared by reaming only without the need to broach the patient's femur).

It would be beneficial to provide optical or electromagnetic tracking for an offset reamer handle to facilitate navigated or robotic-assisted surgical procedures. When tracking instruments in a navigated or robotic-assisted surgical procedure, particularly when using optical tracking, (1) the tracking array must be visible to the camera and (2) the transformation between the tracking array and the point-of-interest must be known. Definition of points-of-interest relative to the tracking array is typically accomplished by either providing a rigid assembly, defining the transform intraoperatively through a calibration/definition step, or if the tool is axisymmetric, allowing the array to rotate about the axis of interest at a set distance. When using an offset reamer in a Total Hip Arthroplasty, a set position is difficult because handle orientation varies relative to the patient and tracking camera. Calibration is not desirable because this adds operative steps and time, and allowing rotation about the tool axis is difficult because the handle is typically offset from the tool axis.

It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some examples, a tracking system for an offset reamer, comprising a clamp to couple with a reamer sleeve of the offset reamer; a coupling coupled with the clamp via a shaft, a tracking array coupled with the coupling, the tracking array comprising target mounts to couple targets with the tracking array, the shaft to fix a distance between the tracking array and an axis of rotation of a reamer element coupled with a driveshaft of the offset reamer; and the targets coupled with the tracking array via the target mounts.

In any preceding or subsequent example, the tracking system further comprising the offset reamer. In any preceding or subsequent example, wherein offset reamer comprises an off-the-shelf (OTS) offset reamer.

In any preceding or subsequent example, the tracking system wherein the clamp comprises an upper clamp portion and a lower clamp portion to clamp to the reamer sleeve of the offset reamer at an axis offset from the axis of rotation of the reamer element.

In any preceding or subsequent example, the tracking system wherein the clamp comprises a nut to fasten the upper clamp portion and the lower clamp portion to the reamer sleeve.

In some examples, an optical tracking system for an offset reamer arranged and configured to facilitate, e.g., a Total Hip Arthroplasty is disclosed. In some examples, the optical tracking system for an offset reamer may include a first mount; a second mount coupled with the first mount, the second mount to couple with optical targets of the optical tracking array, wherein the second mount is rotatably connected to the first mount, a distance between the first mount and the second mount to locate an axis of rotation of the second mount coaxial with a axis of reaming of the offset reamer at a reamer element; and optical targets coupled with the second mount via optical target mounts.

In any preceding or subsequent example, the optical tracking system further including the offset reamer.

In any preceding or subsequent example, the optical tracking system further including a computer system, wherein the computer system includes memory with code, wherein the code, when executed by a processor of the computer system, causes the processor to access to a translation between the optical targets and the reamer element and determine a location of the reamer element at the distal end of the offset reamer.

In any preceding or subsequent example, wherein the first mount includes an upper mount portion and a lower mount portion to clamp to the driveshaft of the offset reamer at an axis offset from the axis of reaming at the reamer element.

In any preceding or subsequent example, wherein the upper mount portion and the lower mount portion each include openings for fasteners.

In some of the preceding or subsequent example, the optical tracking system further including a connector with divots, notches, or other ratchet-like positions, to couple with a shaft of the lower mount portion, the connector to interconnect the second mount to define multiple rotation positions for the optical tracking array about an axis of the shaft.

In any preceding or subsequent example, wherein a second shaft couples the connector with the second mount.

In some examples, an offset reamer with a tracking system. The offset reamer including a proximal end and a distal end, wherein a driveshaft of the offset reamer includes at least one offset portion between the proximal end and the distal end, wherein the distal end of the offset reamer includes a connector for a reamer element; a first mount coupled to the offset reamer between the proximal end and the at least one offset portion; a second mount; and targets coupled with target mounts, wherein the target mounts are coupled with the second mount.

In any preceding or subsequent example, wherein the at least one offset portion includes at least a second offset portion, wherein the second mount is configured to provide an axis of rotation of the target mounts that is coaxial with an axis of reaming of the offset reamer at a connector for the reamer element.

In any preceding or subsequent example, wherein the tracking system includes an electromagnetic tracking system with electromagnetic targets.

In any preceding or subsequent example, the offset reamer further including a computer system, wherein the computer system includes memory with code, wherein the code, when executed by a processor of the computer system, causes the processor to access to a translation between the optical targets and the reamer element and determine a location of the reamer element at the distal end of the offset reamer.

In any preceding or subsequent example, wherein the first mount includes an upper mount portion and a lower mount portion to clamp to a driveshaft of the offset reamer at an axis offset from an axis of reaming at the reamer element.

In some examples of use, a method of reaming a patient's femur via a direct anterior approach is provided. The method including positioning a patient in a supine position. Next, a small incision may be made to access the patient's femur via a direct anterior approach. Thereafter, the curved outer sleeve and the flexible reamer assembly may be inserted into the incision. A surgical drill may be coupled to the proximal end of the offset reamer driveshaft. Thereafter, with the surgeon holding the handle of the outer sleeve, the patient's femur may be reamed as needed.

Examples of the present disclosure provide numerous advantages. For example, utilizing a tracking system with an offset reamer may facilitate navigation assisted surgery or robotic assisted surgery. In addition, the mounting systems for the optical and/or electromagnetic tracking systems may allow the surgeon flexibility in placement of the tracking system mounted to the offset reamer to accommodate various surgical situations such as the position of the patient, placement of the offset reamer, location of the bone for the surgical procedure, and/or the like.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIGS. 1A-1D illustrates perspective views of an example of an offset reamer with optical or electromagnetic tracking;

FIG. 2 illustrates a perspective view of an alternate example of an offset reamer with optical or electromagnetic tracking;

FIGS. 3A-3B illustrate perspective views of an alternate example of an offset reamer with optical or electromagnetic tracking;

FIG. 4 illustrates a perspective view of another example of an offset reamer with optical or electromagnetic tracking with the reamer element in the acetabulum for preparation of the acetabular cup for total hip arthroplasty; and

FIGS. 5A-5D illustrate perspective views of another example of an offset reamer with a handle assembly and an optical or electromagnetic tracking array.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of an offset reamer with a tracking system will now be described more fully herein with reference to the accompanying drawings, in which one or more features of the offset reamer with the tracking system will be shown and described. It should be appreciated that the various features may be used independently of, or in combination, with each other. It will be appreciated that the instrumentation set as disclosed herein may be embodied in many different forms and may selectively include one or more concepts, features, or functions described herein. As such, the offset reamer with the tracking system should not be construed as being limited to the specific examples set forth herein. Rather, these examples are provided so that this disclosure will convey certain features to those skilled in the art.

In accordance with one or more features of the present disclosure, an optical or electromagnetic tracking assembly for an offset reamer is disclosed. In use, as will be described in greater detail herein, the examples couple optical or electromagnetic tracking assemblies to the offset reamer in positions having known translations (stored in the memory of or in memory accessible by a computer system for the tracking assembly) to a position of a reamer element (also referred to as a reamer dome). The reamer dome connects to a driveshaft that has an offset portion. In some examples, an optical tracking array mount couples with the reamer handle or reamer body or reamer sleeve in one or more positions at which a translation between the optical tracking array and the reamer element is known. The translation may refer to coordinates in three-dimensional space such as Cartesian coordinates although such a translation is not limited to any particular coordinate system. In such examples, for instance, an axis for rotation of the optical tracking array is coaxial with the axis for the reamer element or the driveshaft of the reamer element at a connection between the driveshaft and the reamer element. In alternate examples, the optical or electromagnetic tracking array may have a limited number of positions of attachment that each have known translations between the optical tracking array and the reamer element. Note that while the present disclosure illustrates a limited number of different offset reamers, the optical or electromagnetic tracking system assemblies for offset reamers are not limited to the examples illustrated.

Recent developments in navigated surgery and robotic surgery techniques have led to interest in monitoring the progression of surgery and assisting in navigation of surgical instruments during surgery including positioning and orientation of surgical tools with respect to, e.g., a bone. For instruments such as a reamer, different means are employed such as robotically positioning and orienting a sleeve for a reamer to limit movement of the reamer and placing an optical tracking array on the reamer to monitor the position of the reamer via optical means. To implement an optical means (such as one or more cameras coupled with a computer system) for an optical tracking system, the optical means must have an unobstructed line-of-sight view of an optical tracking array on an instrument so the position of, e.g., a reamer element, is known to the computer system. The computer system may include memory with code (or instructions), wherein the code, when executed by a processor of the computer system, causes the processor to access to a translation between the optical targets on an optical tracking array and the reamer element and may determine a location of the reamer element at the distal end of the offset reamer based on the position of the optical targets and the translation. Examples herein advantageously disclose solutions for attachment of an optical tracking array to an offset reamer such that the optical tracking array can be moved to allow an unobstructed line-of-sight view of an optical tracking array by one or more cameras despite the position of the camera(s) and the surgeon. Furthermore, many examples herein facilitate movement of the optical tracking array without necessitating a redefinition or recalibration of the tracking array to a point of interest on the surgical instrument such as a point of interest on, at, or near a reamer element.

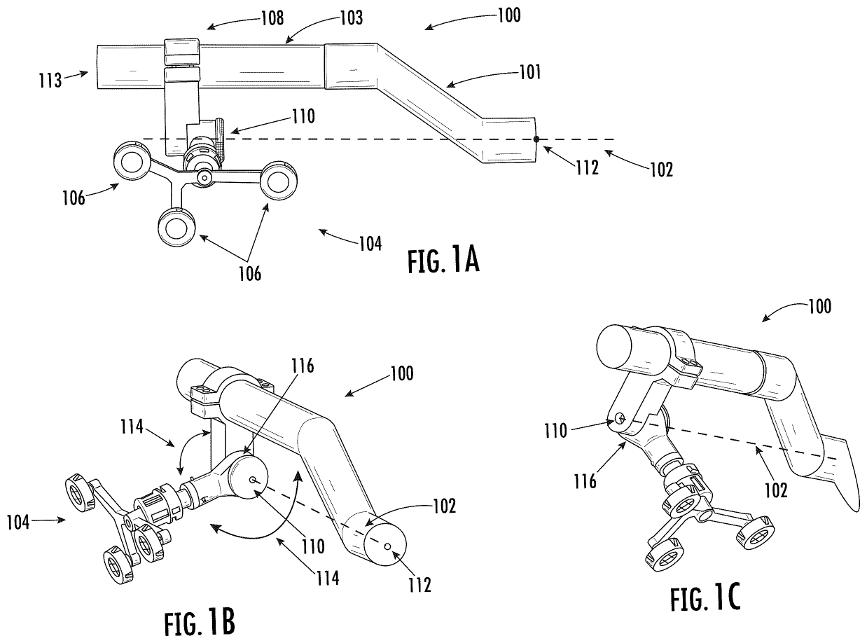

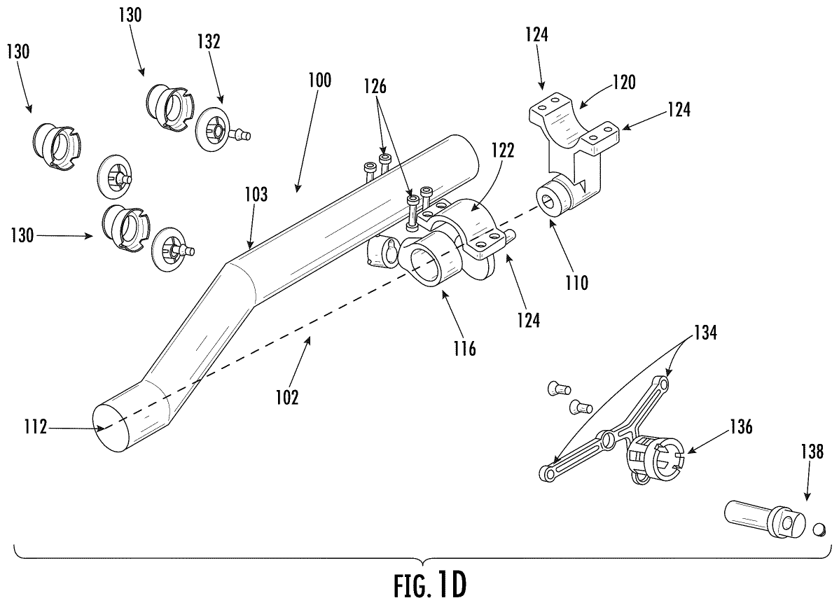

In accordance with one or more features of the present disclosure, with reference to FIGS. 1-3, there are shown different examples of optical tracking arrays coupled with offset reamers.

FIGS. 1A-1D illustrate perspective views of a first example of an offset reamer 100 (or reamer sleeve or reamer handle). FIG. 1A illustrates a first perspective view of the first example of the offset reamer 100. The offset reamer includes offset portion 101 of the driveshaft 103 to place a reamer element at distal end 112. As illustrated, the driveshaft 103 has a proximal end 113 including a coupling for a reamer driver (such as a surgical drill) and a distal end 112 including a coupling for a reamer element (not shown). The coupling for the reamer driver may couple the reamer driver shaft with the driveshaft 103 of the offset reamer 100. The coupling for the reamer element may couple the reamer driveshaft 103 with the reamer element of the offset reamer 100. The driveshaft 103 may include a tubular cover having a rotating element that may be concentric with or otherwise within the tubular cover.

Along the driveshaft 103 and offset from the reamer axis 102 of the reamer element at distal end 112, there is shown a mount 108 for the optical tracking array 104. The mount couples with the driveshaft 103 in a fixed position and attaches with a shaft 110 for rotation of the optical targets 106 (or optical target array) such that the optical targets 106 rotate about the reamer axis 102, coaxially with the axis of the reamer element.

FIG. 1B illustrates a second perspective view of the first example of the offset reamer 100. In FIG. 1B, the optical tracking array 104 can rotate 114 about the shaft 110 having an axis that is coaxial with the reamer axis 102. Given the fixed point of reference of the mount 108 on the driveshaft 103, the fixed axis of rotation 114 of the optical targets 106 about the reamer axis 102, and the fixed position of the optical targets 106 on the optical target array 104, the translation between the optical tracking array 104 and the reamer element at distal end 112 can be determined. Note that determination of the translation may involve adding one or more known distances to coordinates of a point on the optical tracking array 104 along one or more axes (in positive or negative directions) to determine coordinates of a point on the reamer element, which may be in addition to or in lieu of determination of a translation to the distal end 112 of the driveshaft. Note also that, for the purposes of reaming, the rotation of the offset reamer 100 about the reamer axis 102 is not important so the rotation of the optical tracking array 104 is not important, which allows a surgeon to rotate 114 the optical tracking array to any available position about the axis 102 without recalibration or redefinition of the point of interest with respect to the optical tracking array 104.

In some examples, a connector 116 attached to the shaft 110 and the optical tracking array 104 may couple at divots, notches, or other ratchet-like positions, in the shaft 110 and/or in the connector 116 such that the optical tracking array 104 locks into one of multiple predefined positions of the rotation 114 about the shaft 110.

FIG. 1C illustrates a third perspective view of the first example of the offset reamer 100. The mount 108 has an opening at the center of the shaft 110 of the optical tracking array 104. The shaft 110 is coaxial with the reamer axis 102 of the offset reamer 100.

FIG. 1D illustrates an exploded view of examples of components for mounting the optical tracking array 104 to the offset reamer 100. The mount 108 (shown in FIG. 1A) includes an upper mount portion 122 and a lower mount portion 120 that is coupled with the driveshaft 103 (or reamer sleeve or reamer handle) via fasteners 126, such as screws or bolts, and mount openings 124 to fix the position (e.g., coordinates) of the optical tracking array 104 with respect to the reamer element at the distal end 112 of the driveshaft 103. The mount 108 includes a shaft 110 on the lower mount portion 120 to couple with the shaft connector 116. The shaft connector 116 couples with an optical target shaft 138 via a connector 136 of an optical target mount 134. The optical targets 106 (shown in FIG. 1A) include optical elements 130 and optical element fasteners 132 to couple the optical elements at fixed positions with respect to the optical target mount 134.

The optical target shaft 138 can rotate about the shaft 110 on the lower mount portion 120 to rotate the optical targets 106 at a fixed distance from the axis 102 and in a fixed orientation with respect to each other so a computer system may advantageously determine the location of the reamer element. The computer system may include memory or have access to memory with code and data to determine the position (e.g., three dimensional (3-D) coordinates) of the reamer element. The code may include instructions and that data may include a translation (a difference in coordinates or coordinate transformation) between the optical targets 106 and the reamer element. When executed by a processor of the computer system, the code may cause the processor to access the translation between the optical targets and the reamer element and determine a location (coordinates) of the reamer element at the distal end 112 of the offset reamer. In some examples, the location for the reamer element may be determined in a coordinate system associated with a patient subject to a surgery involving the use of the offset reamer 100.

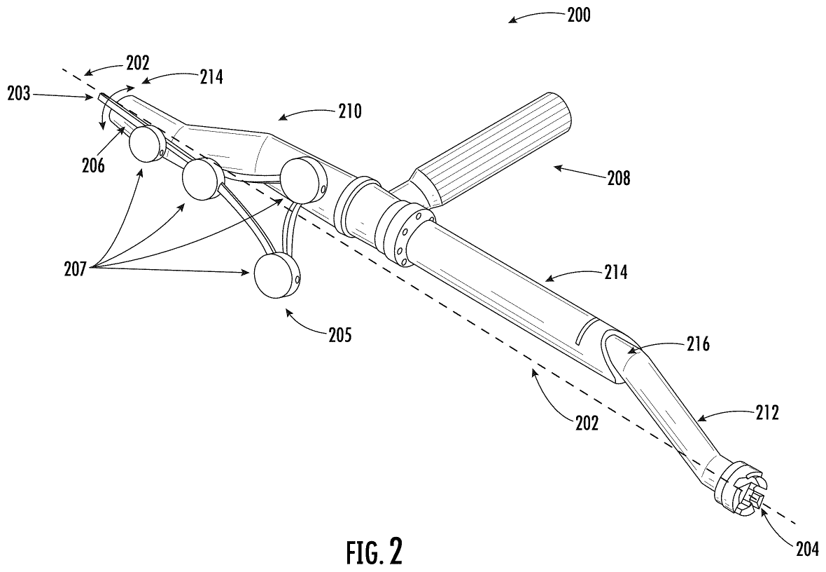

FIG. 2 illustrates a perspective view of a second example of an offset reamer 210 (or reamer sleeve or reamer handle). In FIG. 2, the offset reamer 200 includes a first offset portion 212 of the driveshaft 216 (or reamer sleeve 214) to place a reamer element at distal end 204 and a second offset portion 210 that places a driver (not shown) for the driveshaft 216 on the same axis 202 as, or coaxial with, the reamer element on the distal end 204. As illustrated, the driveshaft 216 has a proximal end 203 including a coupling for a reamer driver and a distal end 204 including a coupling for a reamer element (not shown).

In this example, the offset reamer 200 includes a mount 206 for an optical targets 207 of the optical tracking array 205 and the mount 206 is configured to allow the optical targets 207 to rotate about the axis 202 so that the translation of the locations of the optical targets 207 to the distal end 204 (or a point on the reamer element) is known. In such examples, the optical targets may advantageously be rotated to any available position about the reamer axis 202 without a need for recalibration or redefinition of the point of interest. A computer system may monitor the position of the optical targets 207 via one or more cameras (not shown) to determine the location (e.g., coordinates) of the reamer element coupled with the distal end 204 of the offset reamer 200. For example, the location of the reamer element of the offset reamer 200 may be monitored to facilitate navigation assisted surgery and/or robotic assisted surgery.

In some examples, the optical tracking array 205 may connect to a connector 116 that may couple at divots, notches, or other ratchet-like positions, with a shaft of the mount 206 for the optical tracking array 205 such that the optical tracking array 205 locks into one of multiple predefined positions of the rotation 214 about the axis 202.

The offset reamer 200 may also include a handle 208 fixed to the reamer sleeve 214 to facilitate physical manipulation of the orientation and position of the offset reamer 200 during a surgical procedure.

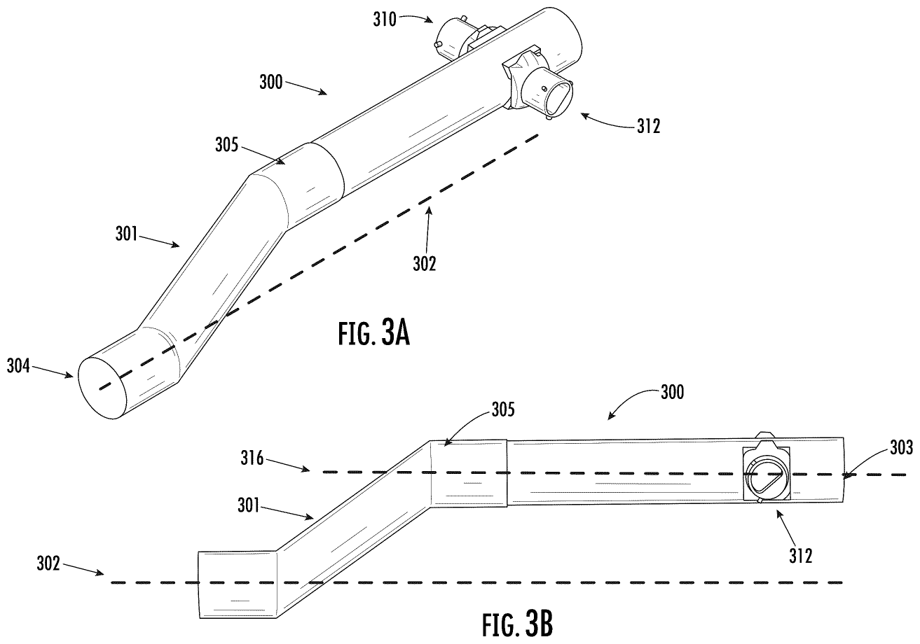

FIGS. 3A-3B illustrate perspective views of a third example of an offset reamer 300 (or reamer sleeve or reamer handle). In FIG. 3A, the offset reamer 300 includes a first offset portion 301 of the driveshaft 305 (or reamer sleeve) to place a reamer element at distal end 304. As illustrated, the driveshaft 305 has a proximal end 303 including a coupling (not shown) for a reamer driver and a distal end 304 including a coupling (not shown) for a reamer element (not shown).

In this example, the reamer element has an axis 302 and offset reamer 300 includes two mounts (310 and 312) for an optical tracking array. The optical tracking system may include an optical tracking array such as the optical tracking array 104 or 205. The optical tracking array may include multiple optical targets but the position of the optical targets is fixed in relation to the mount 310 or the mount 312, depending on the mount selected for the optical tracking system. For instance, a surgeon may determine to connect the optical tracking array to the mount 310 for a surgery. In some circumstances, the surgeon may determine to move the optical tracking array from the mount 310 to the mount 312 for at least part of the surgery.

In such examples, a computer system for the optical tracking system may determine the position of the optical tracking array based on optical recognition of the position of the optical targets, based on surgical procedures established prior to the surgery, or based on other factors. In some examples, the position of the optical tracking array may be defined by informing the computer system by, e.g., selecting the position via a user interface of the computer system. In some examples, the position of the optical tracking array may be defined by identifying a known position (e.g., a divot) with another tracked tool, effectively defining a unique array configuration. For instance, the surgeon may touch the mount 310 or 312, or a divot thereof, with an optically tracked tool to identify the mount on which the optical tracking array is mounted. In some examples, the position of the optical tracking array may be defined by including a fiducial on each potential visible surface in a unique position indicating the orientation of the tool.

A translation for each of the mounts 310 and 312 may be known to the computer system so the computer system may determine the position (e.g., coordinates) of the reamer element or the distal end 304 based on optical images of the optical tracking array captured via one or more cameras coupled with the computer system. With a known position of the reamer element, the computer system can facilitate navigation assisted surgery or robotic assisted surgery.

FIG. 3B illustrates a second perspective of the offset reamer 300 showing a side view of the mount 312 and the distance between the mount 312 and the reamer axis 302. The driveshaft 305 may couple with the reamer element (not shown) with a connector at the distal end 304 and may couple with driver via a connector (not shown) at the proximal end 303.

Note that the number of mounts such as 310 and 312 in the third example is not limited to two mounts. There could be four mounts or any number of mounting positions along the driveshaft 305 of the offset reamer 300. For instance, a ratcheted mount may be attached to the offset reamer 300 to allow the optical (or electromagnetic) targets to rotate to one of several predefined positions about the axis 316. In such examples, the computer system may associate each position with a translation such that when the mounting position of the optical tracking array is known to computer system, the computer system can determine the location of the distal end 304 or a point on the reamer element attached to the driveshaft connector at the distal end 304. In some examples, a touch point probe may point to a divot, notch, or ratchet-like position to identify the mounting position of the optical (or electromagnetic) tracking array. In other examples, a calibration procedure or other procedure may identify the mounting position of the optical (or electromagnetic) tracking array to the computer system.

In another example, an electromagnetic tracking system may be employed on the offset reamer such as the offset reamers 100, 200, and 300 in lieu of or in addition to the optical tracking system. In such examples, an electromagnetic tracking array may be used in lieu of the optical tracking array and the unobstructed line-of-sight view is no longer needed. The computer system may determine a translation of the electromagnetic tracking array to the reamer element based on the known position or mounting position of the electromagnetic tracking array and the known translation between the mounting position and the distal end or the reamer element.

Note that the offset reamers illustrated herein are examples. Examples may use offset reamers of any length and with various different mounting positions for the optical tracking array so long as the computer system can determine the translation between the optical tracking array and the reamer element or a point on the reamer element.

In addition, and/or alternatively, in some examples, the offset reamer may include one or more position sensors such as, for example, sensor arrays, which may be coupled to, for example, the outer sleeve, to track the location of the instrument. Tracking the instrumentation during the femoral reaming workflow may enable live measurement of femoral instrument alignment with respect to the native femoral axis. Tracking this metric may reduce the risk encountered with femoral reaming of puncturing the face of the femur. Thus arranged, the offset reamer may be used in a surgical navigation system. As such, during use, the location, direction, and depth may be tracked to prevent, or at least minimize, over-drilling the patient's bone.

In use, the reamer sleeve can be manufactured from any suitable rigid material such as, for example, any surgical metal such as, for example, titanium, titanium alloys, stainless steel, cobalt-chromium alloys, tantalum, or the like.

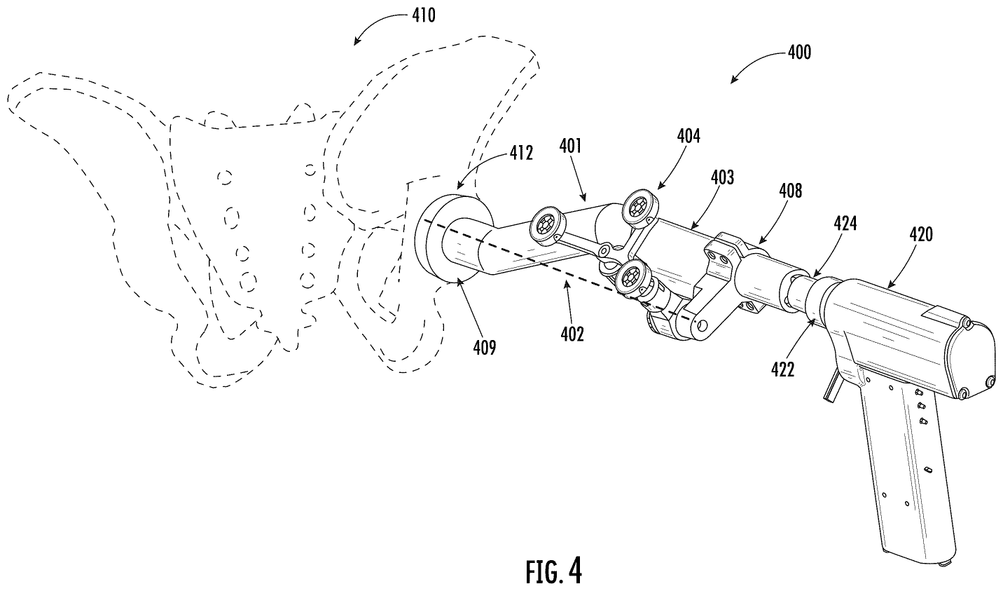

FIG. 4 illustrates a perspective view of another example of the offset reamer 400 with optical or electromagnetic tracking with the reamer element 409 in the acetabulum 412 for preparation of an acetabular cup on a pelvis bone 410 for total hip arthroplasty (THA). During the preparation of the acetabular cup on a pelvis bone 410, the surgeon removes portions of the acetabulum 412 to form the acetabular cup. Formation of the acetabular cup in a proper position is critical to successful total hip replacement. Improper placement of the acetabular cup can lead to increased rates of dislocation, wear, and ion toxicity. The optimal cup position for individual patients depends on differences in functional pelvic position, surgical approach, and femoral anteversion. Note that the offset reamer 400 is similar to the offset reamer 100 shown in FIGS. 1A-1D but other examples may use an offset reamer similar to the offset reamer 200 or the offset reamer 300 for preparation of an acetabular cup on a pelvis bone 410 for THA.

A surgeon may hold a surgical driver 420 coupled with the offset reamer 400 in a position and orientation to place a reamer element 409 on the acetabulum 412 for preparation of the acetabular cup on a pelvis bone 410. The surgical driver 420 includes a driveshaft 422 that couples with a driveshaft 403 of the offset reamer 400 via a coupling 424 to drive the reamer element 409.

A mount 408 for an optical target array 404 couples with the driveshaft 403 of the offset reamer 400 to position the optical target array 404 in a position of rotation about an axis of reaming 402 that does not interfere with the surgeon and is visible to one or more cameras of an optical targeting system.

The driveshaft 403 of the offset reamer 400 includes an offset portion 401 that offsets the axis of reaming from the axis of the surgical driver 420 to the axis of reaming 402. The reamer element 409 couples with a coupling at the distal end of the offset reamer 400 for reaming the acetabulum 412.

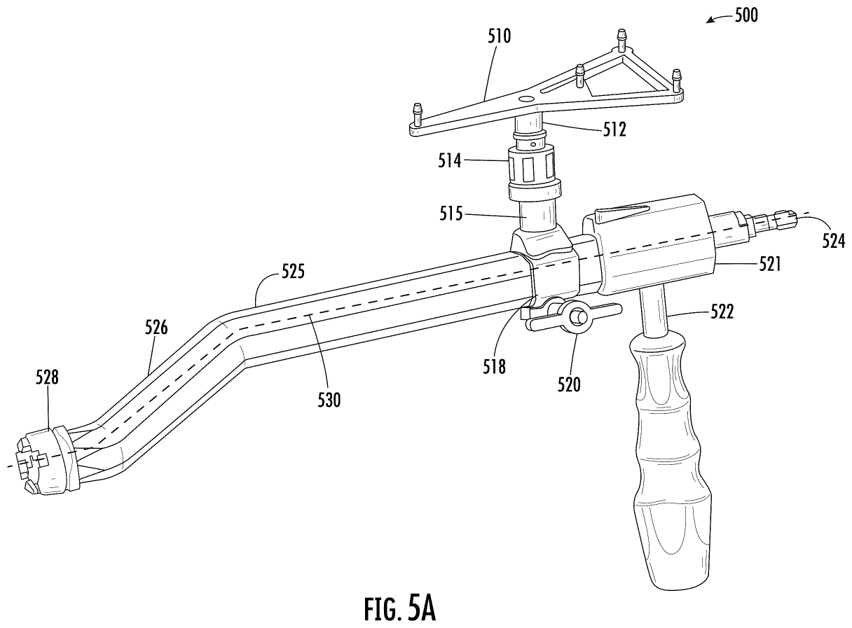

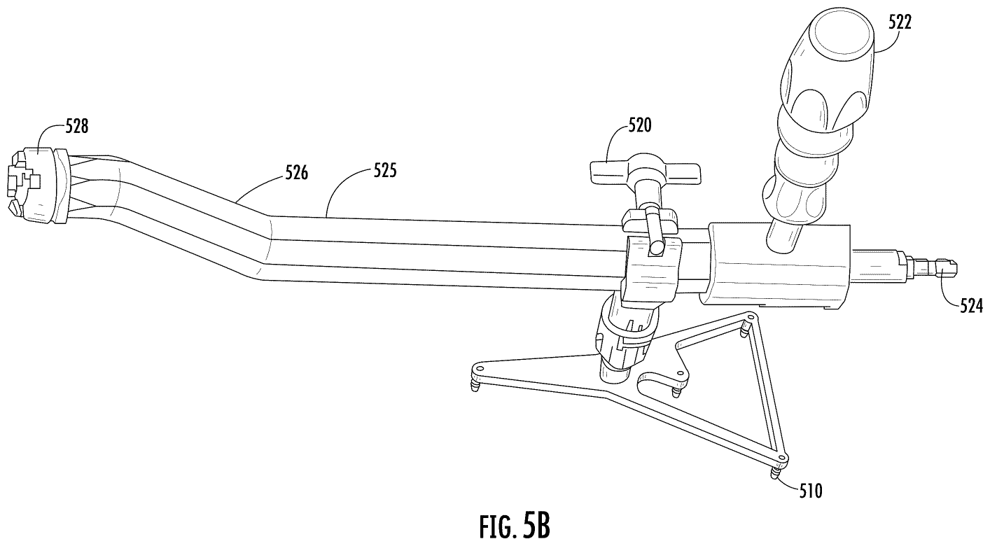

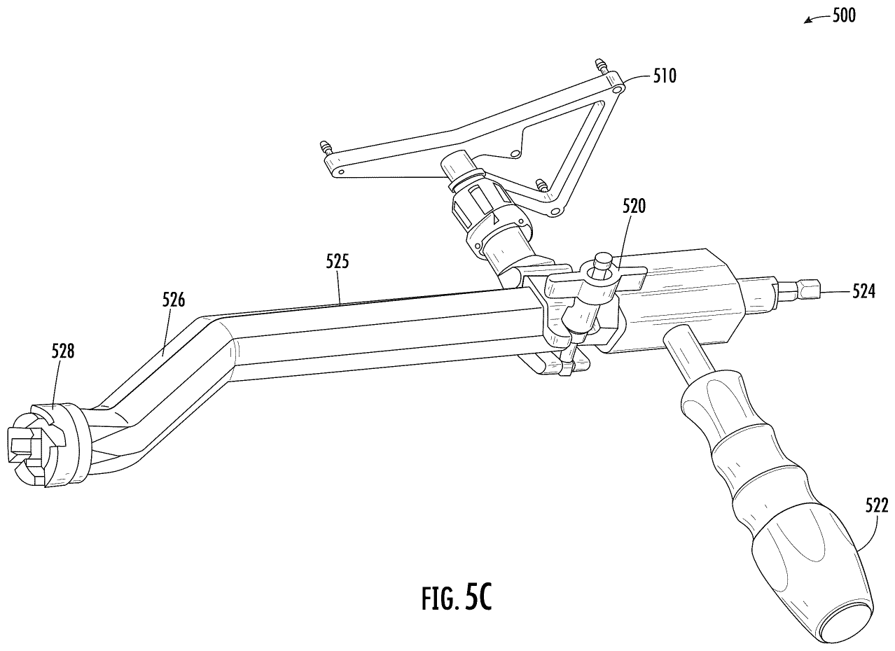

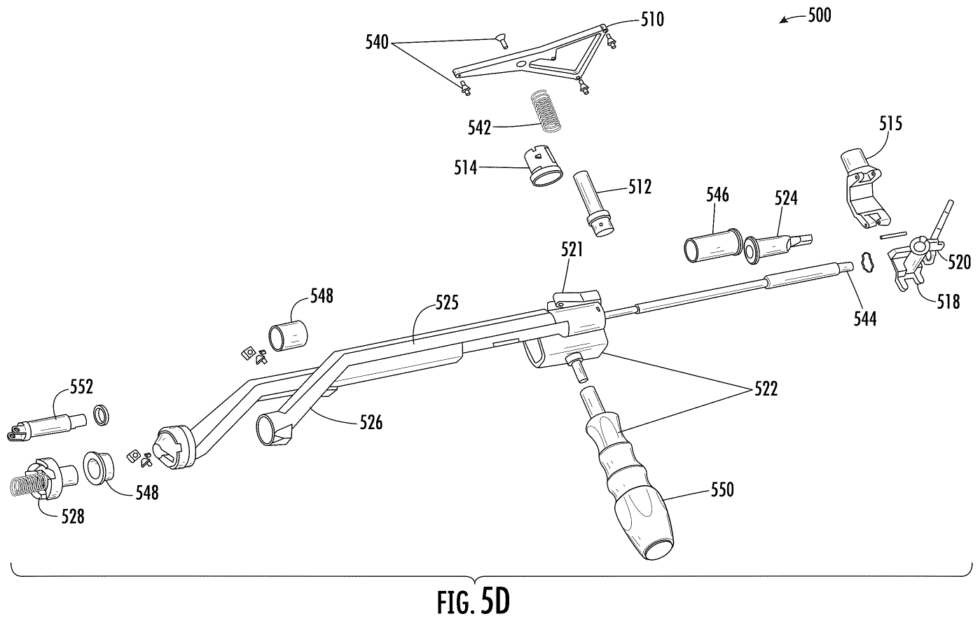

FIGS. 5A-5D illustrate perspective views of another example of an offset reamer 500 with a handle assembly 522 and an optical or electromagnetic tracking array 510. FIG. 5A illustrates a side view of the offset reamer 500 with the handle assembly 522 and the optical or electromagnetic tracking array 510. The offset reamer 500 includes a coupling 524 for a surgical driver (not shown) at a proximal end of the offset reamer 500 and a coupling 528 for a reamer element (not shown) at the distal end of the offset reamer 500. The handle assembly 522 includes a connector 521 to attach the handle assembly 522 to a main portion 525 of a reamer sleeve. The reamer sleeve includes the main portion 525 and an offset portion 526. A reamer driveshaft (not shown) couples the coupling 524 for the surgical driver to the reamer coupling 528 along an axis 530 of the offset reamer 500. As illustrated, in some examples, the reamer sleeve may have a hexagon cross-sectional shape, however this is but one configuration and the reamer sleeve may have any suitable shape such as, for example, circular, polygon, etc. In use, providing a hexagon cross-sectional shape may enable stopping at discrete rotational orientations.

The optical tracking array 510 includes a shaft 512 to couple with a clamp assembly. The clamp assembly includes an upper clamp portion 515, a lower clamp portion 518, and a wing nut 520 to affix or clamp the optical tracking array 510 to the main portion 525 of the reamer sleeve. Note that the upper clamp portion 515 and the lower clamp portion 518 may couple the optical tracking array 510 at any point along the reamer sleeve (525 and 526). In some examples, the clamp (515 and 518) may fasten the optical tracking array at a limited number of different positions about the axis 530 of the offset reamer 500.

Note that, in many examples, the offset reamer 500 may be an off-the-shelf (OTS) offset reamer with or without a handle assembly. In such examples, the offset reamer may require registration. Registration is a process of calculating rigid body transformations between different coordinate systems such as the coordinate system of the offset reamer and the coordinate system of the optical tracking array 510. With the transformations, a computer system may determine, e.g., the position of the reamer element with respect to the optical tracking array 510 and with respect to an acetabulum. The registration process may involve identification of points on the offset reamer 500 and the optical tracking array 510.

In some examples, all possible positions for the optical tracking array 510 may be known and calculations of transformations for an OTS offset reamer 500 may be known. For instance, there may be a distinct number of positions at which the optical tracking array 510 can attach to the offset reamer 500. In some examples, the orientation of the optical tracking array 510 may have indexed positions with respect to the clamp (515 and 518). In some examples, the clamp (515 and 518) may connect to the main portion 525 of the reamer sleeve of the optical tracking array 510 at one of two or more indexed positions on the reamer sleeve. In such examples, after the optical tracking array 510 is attached to the reamer sleeve (525 and 526), a verification process may identify the correct transformations or verify transformations for the current position of the optical tracking array 510. The verification process may require identification of at least one point on the offset reamer 500, the optical tracking array 510, or both.

In any of the examples described herein, an electromagnetic array may be attached to the offset reamer 500 in lieu of an optical tracking array 510.

FIG. 5B illustrates a bottom view of the offset reamer 500 with the handle assembly 522 and the optical (or electromagnetic) tracking array 510. FIG. 5C illustrates another perspective of the offset reamer 500 with the handle assembly 522 and the optical (or electromagnetic) tracking array 510.

FIG. 5D illustrates an exploded view of the offset reamer 500 with the handle assembly 522 and the optical tracking array 510. The optical tracking array 510 includes target mounts 540 to couple the optical tracking array targets (not shown) to the optical tracking array 510 and couples with the upper portion 515 of the clamp via a coupling 514 for the optical tracking array 510, a spring 542, and a shaft 512.

The upper portion 515 of the clamp couples with the lower portion 516 of the clamp about the main portion 525 of the reamer sleeve. The coupling 524 for the surgical driver couples with the reamer driveshaft 544 and spacers 546 and 548 maintain the reamer driveshaft 544 concentric with the upper portion 525 of the reamer sleeve. A third spacer 548 maintains the offset portion of the reamer driveshaft 544 and the coupling 528 for the reamer element (not shown) concentric with the offset portion 526 of the reamer sleeve.

In some examples, the spacer 548 maintains a coupling 552 concentric with the upper portion 525 of the reamer sleeve. The coupling 552 may couple the main portion of the reamer driveshaft 544 with an offset portion of the reamer driveshaft 544. In some examples, the handle assembly 522 includes a connector 521 to couple with or coupled with the main portion 525 of the reamer sleeve and to couple with a handle 550.

In some examples, the handle assembly 522 is integral to the offset reamer 500 or the reamer sleeve (525 and 526). In some examples, the connector 521 is integrated with the upper portion 525 and/or lower portion of the reamer sleeve. In some examples, the upper portion 515 of the clamp and the lower portion 520 of the clamp are part of an integrated clamp assembly. In some examples, an electromagnetic target array is included in lieu of the optical target array and may have a different configuration than the optical target arrays illustrated and described herein.

In accordance with one or more features of the present disclosure, the offset reamer may be used to prepare a patient's acetabular in connection with a Total Hip Arthroplasty, however it should be appreciated that the present disclosure is not so limited and can be used with other implants and/or other surgical procedures such as, for example, insertion of an intramedullary nail. In addition, the present disclosure is not limited to any particular surgical approach and may be used in connection with an anterior approach, a lateral approach, a posterior approach, or any combination thereof. Thus, the present disclosure should not be limited to any particular device and/or procedure unless specifically claimed.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A tracking system for an offset reamer, comprising:
a clamp to couple with a reamer sleeve of the offset reamer;
a coupling coupled with the clamp via a shaft,
a tracking array coupled with the coupling, the tracking array comprising target mounts to couple targets with the tracking array, the shaft to fix a distance between the tracking array and an axis of rotation of a reamer element coupled with a driveshaft of the offset reamer; and
the targets coupled with the tracking array via the target mounts.

2. The tracking system of claim 1, further comprising the offset reamer.

3. The tracking system of claim 2, wherein offset reamer comprises an off-the-shelf (OTS) offset reamer.

4. The tracking system of claim 1, wherein the clamp comprises an upper clamp portion and a lower clamp portion to clamp to the reamer sleeve of the offset reamer at an axis offset from the axis of rotation of the reamer element.

5. The tracking system of claim 4, wherein the clamp comprises a nut to fasten the upper clamp portion and the lower clamp portion to the reamer sleeve.

6. An optical tracking system for an offset reamer, comprising:
a first mount;
a second mount coupled with the first mount, the second mount to couple with optical targets of an optical tracking array, wherein the second mount is rotatably connected to the first mount, a distance between the first mount and the second mount to locate an axis of rotation of the second mount coaxial with an axis of reaming of the offset reamer at a reamer element; and
the optical targets coupled with the second mount via optical target mounts.

7. The optical tracking system of claim 6, further comprising the offset reamer.

8. The optical tracking system of claim 7, further comprising a computer system, wherein the computer system comprises memory with code, wherein the code, when executed by a processor of the computer system, causes the processor to access to a translation between the optical targets and the reamer element and determine a location of the reamer element at the distal end of the offset reamer.

9. The optical tracking system of claim 6, wherein the first mount comprises an upper mount portion and a lower mount portion to clamp to a reamer sleeve of the offset reamer at an axis offset from the axis of reaming.

10. The optical tracking system of claim 9, wherein the upper mount portion and the lower mount portion comprise openings for fasteners.

11. The optical tracking system of claim 9, further comprising a connector with divots, notches, or other ratchet-like positions, to couple with a shaft of the lower mount portion, the connector to interconnect the second mount to define multiple rotation positions for the optical tracking system about an axis of the shaft.

12. The optical tracking system of claim 11, wherein a second shaft couples the connector with the second mount.

13. An offset reamer with a tracking system, comprising:
the offset reamer comprising a proximal end and a distal end, wherein a driveshaft of the offset reamer comprises at least one offset portion between the proximal end and the distal end, wherein the distal end of the offset reamer comprises a connector for a reamer element;
a first mount coupled to the offset reamer between the proximal end and the at least one offset portion;
a second mount; and
targets coupled with target mounts, wherein the target mounts are coupled with the second mount.

14. The offset reamer of claim 13, wherein the at least one offset portion comprises at least a second offset portion, wherein the second mount is configured to provide an axis of rotation of the target mounts that is coaxial with an axis of reaming of the offset reamer at a coupling for the reamer element.

15. The offset reamer of claim 13, wherein the tracking system comprises an electromagnetic tracking system with electromagnetic targets.

16. The offset reamer of claim 13, further comprising a computer system, wherein the computer system comprises memory with code, wherein the code, when executed by a processor of the computer system, causes the processor to access to a translation between the optical targets and the reamer element and determine a location of the reamer element at the distal end of the offset reamer.

17. The offset reamer of claim 13, wherein the first mount comprises an upper mount portion and a lower mount portion to clamp to the driveshaft of the offset reamer at an axis offset from a driver shaft axis at the reamer element.

18. The offset reamer of claim 17, wherein the upper mount portion and the lower mount portion comprise openings for fasteners.

19. The offset reamer of claim 17, further comprising a second connector with divots, notches, or other ratchet-like positions, to couple with a shaft of the lower mount portion, the second connector to interconnect with the second mount to define multiple rotation positions for the tracking system about the axis of the shaft.

20. The offset reamer of claim 19, wherein a second shaft couples the second connector with the second mount.
